(19) United States
(12) Patent Application Publication
(10) Pub. No.: US 2009/0018475 A1
(43) Pub. Date: Jan. 15, 2009

(54) UV-Curable Dressing

(76) Inventor: Christophe Lesca, Paris (FR)

Correspondence Address: OLIFF & BERRIDGE, PLC, P.O. BOX 320850, ALEXANDRIA, VA, 22320-4850, US

(21) Appl. No.: 12/216,599

(22) Filed: Jul. 8, 2008

(30) Foreign Application Priority Data
Jul. 10, 2007 (EP) 07360031.4

(51) Int. Cl. A61F 5/00; A61F 15/00

(52) U.S. Cl. 602/2; 602/54; 602/57

(57) ABSTRACT

Dressing combining a flexible membrane with a chemical available in a state able to impregnate the membrane and whose curing is controlled. Curing of said substance is effected by UV radiation.

The present invention relates to a dressing that can be used in surgery, combining in particular a sterilizable fabric with a chemical whose curing is controlled.

The combination of such fabrics and adhesives curable for example on contact with air is already known. However, in this case there is no control over the time factor: as soon as the fabric is impregnated with the self-curing adhesive, the surgeon must place it on the application site with no loss of time before it hardens and makes the operation impossible.

The goal of the present invention is to be no longer dependent on the time factor, or at least to control this time constraint better while handling the dressing. The use of a classical cyanoacrylate adhesive, which polymerizes in its viscous consistency in one to one and a half minutes, does not allow dressings to be used in all their potential applications because of the restricted time window they offer.

Furthermore, another goal of the invention is to adapt as well as possible to certain constraints linked to the utilization sites of these dressings in order to ensure maximum curing efficacy regardless of the type of application.

For these purposes, the dressing according to the invention, combining—as stated—a flexible membrane with a chemical substance available in a state able to impregnate the membrane, curing of which is controlled, is characterized in particular by the curing of said substance being effected by UV radiation.

The main advantage of this treatment is the following: the use of such a substance affords freedom from all time constraints, the user being in control of the time factor under all circumstances by deciding on the time of polymerization.

Said substance can thus be a UV acrylic adhesive.

Alternatively, curing can be effected by polymerization with dual stimulation:
by steam;
by UV radiation.

The usefulness of this dual stimulation is the following: exposure to ultraviolet radiation alone has the disadvantage that the rays do not necessarily in all cases reach the deep part of the dressing, in contact with the covered mucosa, and polymerization of the adhesive is hence not ensured at these sites.

The use of the steam component, on the other hand, enables polymerization of the adhesive in contact with the mucosa to occur due to the presence of blood. The UV rays control the curing of the surface part of the dressing at the end of the procedure to seal the entire dressing.

The two actions combine, reinforcing the efficacy of the dressing.

The problem of ultraviolet rays acting in depth at the surfaces covered by the dressings is all the more acute when the membrane, for example a cellulose gauze, is thick. In this case, it is entirely possible, according to one optional protocol, to pass the deep face of the dressing under the ultraviolet radiation source for a fraction of time in order to start polymerization on this face. Such a step would have the additional advantage of beginning to stiffen the dressing so that it conforms more readily to the site to be covered.

Polymerization then completes at this level because of the moisture present, in particular, in the blood.

According to one option, the substance in question can be an adhesive of the cyanoacrylate type. Alternatively, it can be a substance based on polymerizable silicone, either solely by exposure to ultraviolet radiation or to the aforementioned dual stimulation. This substance may include at least one activator sensitive at one wavelength, or preferably several activators. Thus, according to one possible example, it can include three activators responding to three different wavelengths, which facilitates its use and enables it to be used with classical ultraviolet radiation sources that are easy to procure and are available if desired for other applications.

One of the basic points linked to the utilization of the dressing according to the invention resides in the possibility of using it extemporaneously. Thus, the membrane and the curing substance can, for the purpose, be packaged in a kit comprised of two individual chambers separated by a partition enabling said substance to pass into the membrane chamber when the substance is expelled from its own chamber.

The entry of the adhesive, or more generally the curing substance, into the area containing the membrane enables the latter to be sufficiently impregnated before the rest of the kit is opened for immediate use of the dressing. Preferably, the chamber of the curing substance is delimited by light-opaque walls to guard against any risk of untimely polymerization.

More specifically, the kit can consist of two blister packs made of flexible material joined by a partition allowing the curing substance to pass from one blister to the other by tearing or permeability, the expulsion of said substance from one to the other being effected by pressure on the blister of the curing substance.

The use of a flexible material for the blister packs then enables the membrane to be impregnated by manipulating its blister pack.

The use of such a double blister pack is useful for several reasons:
the frequently encountered problem of expiration dates of reconstituted products would be very easily resolved by the fact that the closest date would be the determinant date in the specific case;
at the present time, the effects of this type of adhesive interacting with an oxidized cellulose membrane in the course of time are unknown. Since preparation takes place extemporaneously, this problem no longer arises;
finally, it is far more difficult to package a membrane with a cyanoacrylate adhesive under vacuum, excluding all steam to prevent polymerization from starting. On the other hand, it is relatively easy to package the dry membrane in a sufficient vacuum.

Alternatively, the kit can be a flexible bottle with an end fitting enabling the adhesive to be applied directly to the fabric, either in its blister pack or after it has been cut.

The invention also relates to a method for preparing a dressing meeting the above conditions, consisting of:
removing the membrane from the chamber of its package;
cutting it to the size required by the planned application;
replacing the membrane in its chamber;
expelling the curing substance from its chamber so that it penetrates into the membrane chamber; and
manipulating the membrane chamber to effect the impregnation.

It is more difficult to cut out an already-glued fabric because of the risk of surgical instruments sticking to it during the manipulation. Instead of offering a dressing cutting method with disposable instruments, which complicates the maneuvering and packing of the product and is uneconomical, cutting is done before impregnation. The placement of the dressing at the application site can then be done by bringing the dressing to the site to be protected with the fingers, or with surgical clamps, keeping it applied by finger movements until polymerization begins. The dressing is next sutured, then exposed to ultraviolet radiation to cure the dressing. If blood is still oozing, additional adhesive can be applied directly to the dressing, for example using the bottle plus end fitting, or spread with the fingers, before spontaneous or ultraviolet polymerization.

Among the contemplated applications, it is possible to join two blood vessels whose cut edges are kept in contact with each other with the aid of a pre-glued membrane according to the invention. One suture, or even two, can be positioned to bring the two edges together. The glued dressing, girdling the anastomosis, would then be applied to ensure that the assembly is sealed.

It is also possible to use a membrane and a curing substance according to the invention for a limited time in the following application: the membrane is made of a transparent, colorless fabric designed to enable time-limited skin traction. Thus, it could be possible to cause an invisible, colorless, non-resorbable membrane to adhere to the skin of the back of the neck, at the hairline, behind and below the ears.

Small tabs of this membrane, possibly protected from the adhesive to begin with, could extend rearward to the scalp and be connected to each other, at the rear of the scalp, by a colorless thread to draw back the skin of the neck rearward. This thread or threads would then be clipped to the tabs.

Such a device could, in the course of one evening, meet the needs of a woman who has rejected plastic surgery of the neck. Another application could be envisaged in the area of creating superficial skin tension.

At the present time there are dressings with adhesive on one face (not cyanoacrylate adhesive) that can cover small sores, replacing skin suture stitches: these are dressings known by the trade name Steri-Strips. Small tabs of fabric—not absorbable in this case—could be embedded in the cyanoacrylate type adhesive and, once removed from their blister pack, could cover a sore in the manner of said Steri-Strip dressings. The drawback is that the edges of a sore have to be kept in contact with each other until polymerization occurs. The advantage is that the seal is far more effective and durable over time.

According to another example, the invention could be used to treat fractures with an adhesive fabric. This fabric is then positioned over the bone edges of the fracture once it is reduced. In this case, the membrane has to be relatively thick so that it becomes rigid once the adhesive polymerizes. The stiffened membrane adhering to the bone then has the properties of a metal plate for osteosynthesis. It could also be screwed to the bone.

This device, not resorbable by the body, should in principle be removed once consolidation is complete.

Of course, the invention allows other applications that cannot be described exhaustively.

1. Dressing combining a flexible membrane with a chemical available in a state able to impregnate the membrane and whose curing is controlled, wherein the curing of said substance is effected by ultraviolet radiation.

2. Dressing according to claim 1, wherein said substance is a UV acrylic adhesive.

3. Dressing according to claim 2, wherein the curing of said substance is effected by polymerization with dual stimulation:
by steam;
by ultraviolet radiation.

4. Dressing according to claim 3, wherein said substance is a cyanoacrylate type adhesive.

5. Dressing according to claim 1, wherein the substance is based on polymerizable silicone.

6. Dressing according to claim 1, wherein the substance includes at least one activator sensitive to one wavelength.

7. Dressing according to claim 6, wherein the substance includes three activators responding to three distinct wavelengths.

8. Dressing according to claim 1, wherein the membrane and the curing substance are packaged in a kit comprised of two individual chambers separated by a partition allowing passage of said substance to the membrane chamber when it is expelled from its own chamber.

9. Dressing according to claim 8, wherein the chamber of the curing substance is delimited by walls opaque to light.

10. Dressing according to claim 8, wherein the kit comprises two blister packs made of flexible material joined by a partition allowing the curing substance to pass from one blister to the other by tearing or permeability, the expulsion of said substance from one to the other being effected by pressure on the blister of the curing substance.

11. Dressing according to claim 10, wherein impregnation of the membrane is effected by manipulation of its blister.

12. Dressing according to claim 1, wherein the membrane is made of cellulose gauze.

13. Method for preparing a dressing according to claim 8, comprising:
removing the membrane from the chamber of its package;
cutting it to the size required by the planned application;
replacing the membrane in its chamber;
expelling the curing substance from its chamber so that it penetrates into the membrane chamber; and
manipulating the membrane chamber to effect the impregnation.
